(19) United States
(12) Patent Application Publication
(10) Pub. No.: US 2017/0367799 A1
(43) Pub. Date: Dec. 28, 2017

(54) Oral irrigation device

(71) Applicant: Terence John Mullin, Orange, CA (US)

(72) Inventor: Terence John Mullin, Orange, CA (US)

(21) Appl. No.: 14/999,754

(22) Filed: Jun. 24, 2016

(51) Int. Cl. A61C 17/02; A61M 3/02; A61C 1/08; A61H 13/00

(52) U.S. Cl. CPC A61C 17/0202; A61C 17/0214; A61H 13/005; A61M 3/0279; A61C 1/088; A61M 2205/586; A61M 2205/587; A61M 2205/59; A61M 2205/0216

(57) ABSTRACT

The oral irrigation/liquid delivery device provides flow controlled fluid to body areas specifically the mouth, or parts of body or other non-body areas via pump device or connection to water source through a wand handle with bulbous smooth head with multiple openings/orifices for the fluid to flow to ensure that no high-pressure jets result (unless within the design) and can be used in confined area; idea for irrigating or rinsing the mouth after jaw surgery or other situation where it is not possible or recommended to use a high-pressure “water pick” type of device; allows user who may have a problem sucking in water to rinse to have fluids safely delivered to mouth to clear and clean it; allows for the delivery of medicated and or other liquid delivery of water or any other liquid to rinse or even imbibe; said device could also deliver feeding fluids; optional connection via any pump or faucet diverter system to delivery fluids, including option to have a siphon mix of one solution along with another with the secondary solution being introduced to the flow as a result of the pump mechanism or a siphon mechanism based upon the flow of a fluid that is the primary solution. Optionally, a rubber or other suitable tube or hose may be connected between the wand and the tip/connecting point to the pump or fluid delivery device. The same device or its components could be incorporated into other devices such as water bottles or syringe ends.

### CROSS-REFERENCE TO RELATED APPLICATIONS

This application is based on and claims the benefit of U.S. Provisional Application No. 62/184,366, which was filed Jun. 25, 2015 with confirmation number 6985, the entirety of which is hereby incorporated by reference as filed in the United States. The application number from the latest correspondence calls out application No. 14/999,754.

Inventor is Terence Mullin, 1388 N. Kennymead Street, Orange, Calif. 92869 USA ph. 949.697.2139

### BACKGROUND OF THE INVENTION

### Field of the Invention

The present invention relates to a device to provide flow controlled water or irrigation/feeding fluids to the oral cavity or mouth via pump devices or water bottle or syringe devices or connection to fluid source through a wand handle with bulbous smooth head with multiple openings for the water to flow to ensure that no high-pressure jets result (unless within the design) and can be used in confined area. The invention provides for rinsing the mouth or the delivery of necessary fluids into the mouth after jaw surgery or other situation where it is not possible or recommended to use a high-pressure “water pick” type of device; allows user who may have a problem sucking in water to rinse to have water safely delivered to mouth to clear and clean it. The invention allows for the delivery of medicated and or other liquid delivery of water or any other liquid to rinse or even imbibe. The invention can also be used to allow for the delivery of other fluids, including feeding fluids safely without the possibility of damage caused by other pointed or sharper tubes. The invention incorporates optional connection ends for any pump or syringe or faucet diverter system to delivery fluids, including option to have a siphon mix of one solution along with another with the secondary solution being introduced to the flow as a result of the pump mechanism or a siphon mechanism based upon the flow of a fluid that is the primary solution. Optionally, a rubber or other suitable tube or hose may be connected between the wand and the tip/connecting point to the pump or fluid delivery device. This same bulbous end of the invention can be incorporated into other uses including water bottles or syringes with affixed straws/tubes etc.

One of the most common “don'ts” after oral surgery, or for under six years of age or patients with a disability or diminished motor skills is “Do not use drinking straws, a water pik high pressure, sharp-tipped oral hygiene device for weeks after the surgery unless otherwise advised by your doctor.” Whereas there are devices that provide high-pressure irrigation, none provide “safety designed” devices such as this invention to allow for its use in the cases mentioned. It is critical that the patient takes care not to tear sutures with sharp objects or brushes or high-pressure “water pick” pump devices.

### SUMMARY OF THE INVENTION

Accordingly, there is a need in the art for an effective and safe system means of providing oral irrigation using existing pump systems and passive fluid delivery systems that eliminate sharp or hazardous tubes or straws or “pick ends” in addition to reducing or eliminating high-pressure jets of fluid into the mouth that may cause potential discomfort or damage to the user's mouth.

In accordance with one embodiment, the present invention provides a system for oral irrigation without a high-pressure, potentially damaging water stream. This is critical for patients who undergo orthognathic procedures to correct medical issues. According to NIH estimates, it is suggested that over 10 million Americans are affected with Temporomandibular joint and muscle disorders and require surgery. Second to orthognathic surgery is the 10 million wisdom teeth which are removed each year. With an average patient having two teeth removed, that represents five million people who have wisdom teeth removed. Third, 74% of all adults have had a tooth extracted. After that procedure, care must be taken not to damage the surgical site, while at the same time, keep the site clean. Inclusive of the wisdom teeth extractions, there are an estimated 20 million extractions per year, comprised of 5 M wisdom teeth extractions and 15 M general extractions. In all of these cases, two instructions are clear: no high-pressure water pick like irrigation and do not insert any cleaning device into your mouth with a pointed or sharp tip.

In one embodiment, the device incorporates a rounded tip end with multiple openings to allow the flow of fluid to irrigate the mouth. This rounded tip may be made of a rubber-like material to further mitigate any potential damage to soft or healing tissues.

Yet another embodiment would incorporate a flexible, shock absorbing shaft to further mitigate potential damage caused if the device is pushed too hard into the mouth, or if the user's hands were “shaky” and could potentially cause undue stress of the device in the mouth.

In accordance with another embodiment, the device's rounded, multi-opening bulbous tip allows for the flow of water or irrigation fluid even if some parts of the bulbous tip were pressed against the soft tissue of the mouth and blocked the flow of fluid.

In one embodiment the handle incorporates an expansion chamber that also allows water pressure to “buffer” in order to not have high pressure release of water (or other solution as prescribed) that is typically associated with commercially available oral irrigation pumps. This ensures that the flow of fluid does not cause damage while at the same time does not require any modification to the existing pump system.

Yet another embodiment the device can be produced with or without an optional, unique anti-drip collar that limits or stops any water from flowing down the device while in use in the user's mouth.

In a further embodiment, the device will be constructed out of a type of plastic to allow for both sterilization and sterile packaging and will support the typical post-surgical recommendation is to use warm salt water and or an antibacterial agent (Chlorhexidine, or Listerine or sterile saline) to irrigate the mouth.

In accordance with a still further embodiment, this same invention could be configured to fit the end of a water bottle or syringe to allow for use without a pump making it ideal for use while not at home.

In another embodiment, the same water bottle or syringe use could be used to aid in post-procedure feeding of prescribed liquid diets. The invention would allow for the safe insertion of the device to allow the user to deliver the liquid without fear of accidentally causing damage or having a single hole (like a straw) be blocked and restricting the flow of fluids.

In still another embodiment, utilizing a common “squeeze bottle”/sports bottle for feeding that would support “thicker fluids,” our safety tip feeding bottle has not only the advantage of our patent-pending tip, but allows for easier use. Using our invention's design, the water bottle implementation is less “medical looking” for use in public and can be branded to look like other “squeeze bottles” that have been made common place by the “sports use” markets. Additionally it is easier to clean and maintain as it has no moving parts and does not suffer from the failure of the plunger that accompanies all syringes. Our same patent-pending solution would be ideal for “oral rinsing” and as a water bottle as a dual-purpose solution as well. Through our patent-pending invention with safety tip, tremendous potential licensing agreements could be reached with other so-called “sports bottle” manufacturers who would now want a safety tip.

A yet further embodiment based upon the post-surgical irrigation and fluid delivery designs, the pre age 6 version mitigates and eliminates the “rigid tipped/rigid wand device” restrictions of use. These same advantages could be extended to the elderly or invalids.

In a still further embodiment, by incorporating the larger version of the invention with varying lengths based upon use cases, the “ease of use” by children, elderly or invalids could be increased. Depending upon the use case, the invention itself being of a larger diameter than the standard tip could be made of a flexible, shock absorbing, bendable material that would still accomplish the structural and water flow characteristics necessary to function. Based upon its flexibility, the chances of accidental damage caused by a rigid wand could be mitigated.

By incorporating some or all of the recommendations stated above, depending upon the use case, the overall potential product safety issues associated with inserting a potentially rigid, pointed, or non-flexible device into a users' mouth are potentially reduced.

In still another embodiment, the overall design of the invention's wand/tip could be modified in color and or shape. Additionally, the addition of a battery powered LED providing a “light pipe” to the shaft of the wand would add an additional “attraction” to the use in addition to providing added visibility of the child's success in oral hygiene. (Think of the “Harry Potter” magic wand for oral hygiene angle or the Star Wars light saber angles). In this use case, the product adoption for children could be potentially increased.

In still another embodiment, there are many conditions that make typical oral hygiene difficult, especially considering the use of a water pick type of device. Individuals that are any type of paralysis, palsy, or other debilitating conditions where the user does not have the manual dexterity to safely use a water pick device as it could be either unsafe or not possible. They all will benefit from the abilities wand. Additionally, by virtue of its design, care givers will be able to use the abilities wand to assist in its intended uses.

In still another embodiment, an optional “flexible” or accordion shock absorbing section of the wand could be incorporated to further reduce “impact issues” of the wand being pushed accidentally hard or being “bent” at an angle that could cause discomfort or harm.

In still another embodiment, an optional suction capability could be incorporated whereby a suction line(s) could be incorporated into either fixed or “sliding” collar(s) that the user would hold in place with their lips while the wand is move in and around the oral cavity. Either an organic flushing of the irrigation solution would take place (by the user's own blowing of the solution out) or it could be aided by an optional suction pump device. With this option, those bedridden or unable to position themselves over a sink or emesis basin could still take advantage of the OTC pump oral hygiene regime.

In still another embodiment, any combination of the optional configurations could be manufactured in the device, such as adding the no-drip collar with the flexible/shock absorbing shaft etc. The same would stand for adding the bulbous tip onto other fluid delivery options such as syringes water bottles etc.

### BRIEF DESCRIPTION OF THE DRAWINGS

FIG. 1A is a schematic illustration of the device with one embodiment.

FIG. 1B illustrates an embodiment of the device with the optional no-drip collar.

FIG. 2 is an enlarged/exploded view of one embodiment of the device's bulbous round tip end.

FIG. 3A illustrates an embodiment of the device with the optional flexible/shock absorbing shaft.

FIG. 3B illustrates an embodiment of the device with the optional draining adapter.

### DETAILED DESCRIPTION OF PREFERRED EMBODIMENTS

Reference will not be made in detail to particular embodiments of the bulbous tip and its incorporation onto the oral irrigation/fluid delivery device, one or more examples of which are illustrated in the drawings. Each embodiment is presented by way of explanation of the invention, and not as a limitation of the invention. For example, features illustrated or described as part of one embodiment may be used with a different embodiment to yield still another embodiment. It is intended that the present invention encompasses these and other modifications and variations as come within the scope and spirit of the invention.

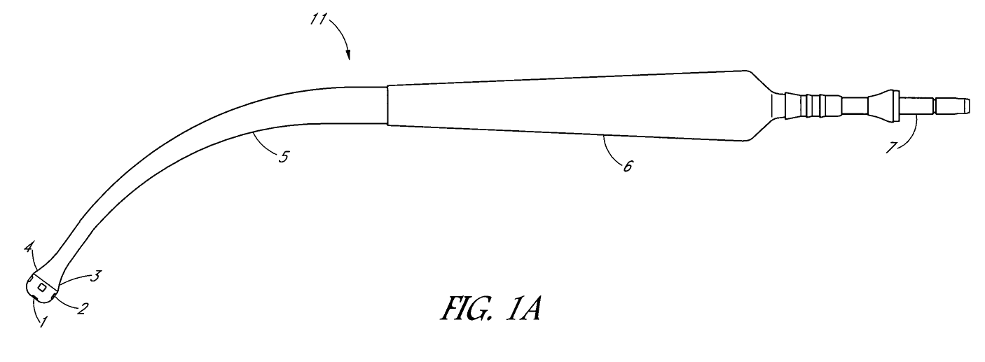

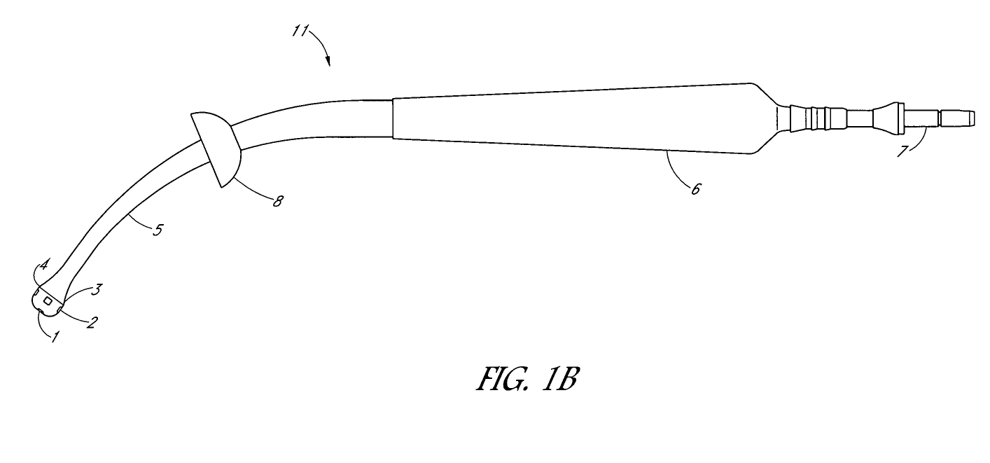

FIGS. 1A through 1B illustrate an embodiment of the device 11 in accordance with the invention. The device 11 is particularly suited as an oral irrigation or fluid delivery system, in cases of post-surgical procedures, or ability to use a device of this type safely is recommended. The device 11 includes an elongated delivery tube 5 having a distal end 3 and a proximal end 7. As used herein, “distal” refers to the direction closest to the user or patient, i.e. in their mouth and “proximal” refers to the connecting point of the device to a fluid source.

The proximal end 7 of the oral irrigation device 11 is configured with any conventional fluid delivery system, being an oral irrigation pump, syringe, water bottle of the like. The oral irrigation device 11 may be configured directly with the pump, syringe or water bottle, or may be connected/integrated with the fluid delivery source through any manner or configuration of intermediate members. Such intermediate members may include any desired functionalities, such as the ability to regulate the flow volume and or pressure through the device.

The device 11 includes an optional no-drip collar 8. The optional no-drip collar 8 is attached to the shaft 5 of the device 11 as illustrated in FIG. 1B.

In a particular embodiment of the device 11 illustrated in the figures, a handle member 6 is provided at the proximal end 7 of the device 11. The handle member 6 may encompass the device 11, or include “adapters” that mate the device with the fluid delivery mechanism of choice (direct fluid delivery, water pick like pump, syringe, water bottle etc.), as illustrated in FIGS. 1 through FIGS. 3. The handle member 6 can also include a fluid “expansion chamber” that will essentially buffer the pulsing of water and optionally lowering the pressure of the fluid being delivered by the device. The handle member 6 is connectable to any commercially available or designed source for supplying liquid to the device 11. Although not illustrated, it should be understood that the handle 6 may include any manner of functional features, such as a valve or other device to regulate the fluid flow and pressure through the device 11. Desirably, the handle member 6 includes any suitable adapter 7 for connecting the device 11 to fluid delivery source or to any other intermediary member. The adapter 7 may be configured as a universal adapter for insertion into a wide diameter range of external fluid delivery systems or tubing or syringe or water bottle. In an alternative embodiment, the adapter 7 may comprise any type of quick release mechanism that mates the device 11 to a fluid delivery source or other member. The adapter 7 may include any manner of internal structure to minimize leakage through the connection. For example, the adapter 7 may include an adapter to connect to a commercially available WaterPik or other commercial oral water flossing device.

Referring to FIG. 1B, the optional no-drip collar 8 which is affixed to the device's shaft 5, would aid in preventing excess or expelled fluid from running down the device's 11 shaft 5. The no-drip collar 8 would divert the fluid to “fall” over a desired location such as a sink or basin of some sort.

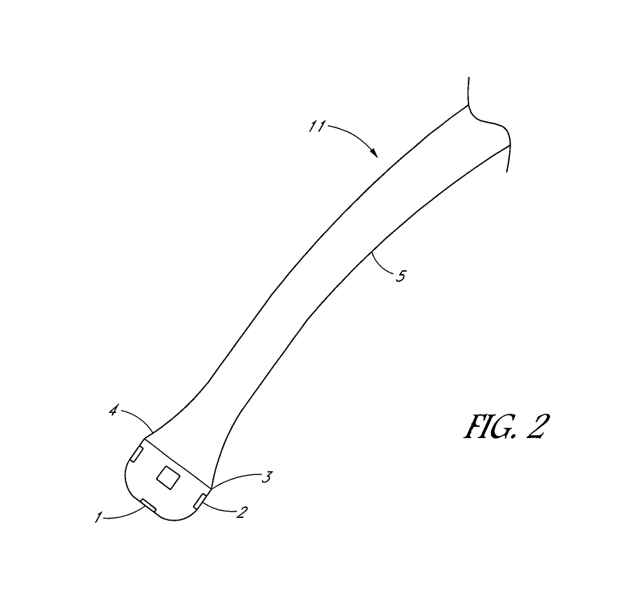

Referring to FIGS. 1 through FIGS. 3, the bulbous tip 3 of the oral irrigation device 11 may take on any desired rounded or smooth shape or configuration. For example, referring to FIG. 2, the exploded/enlarged view shows that the tip is comprised of a single orifice 1 at the top of the tip, four or more (depending upon the diameter of the designed tip) orifices surrounding the tip's circumference 2 that can be configured to direct fluid directly out of the bulbous tip or deliver the fluid at an angle to cause for a circular disbursement of fluids 4. Different embodiments of the bulbous tip 3 that are not illustrated would be the incorporation of the device tip onto the end of a water bottle or syringe for oral feeding or oral irrigation uses. The design of the device not only provides non high pressure delivery of fluids to the user but also presents a rounded non-irritating surface device end (bulbous tip 3) and minimizes any discomfort or potential injury to the patient.

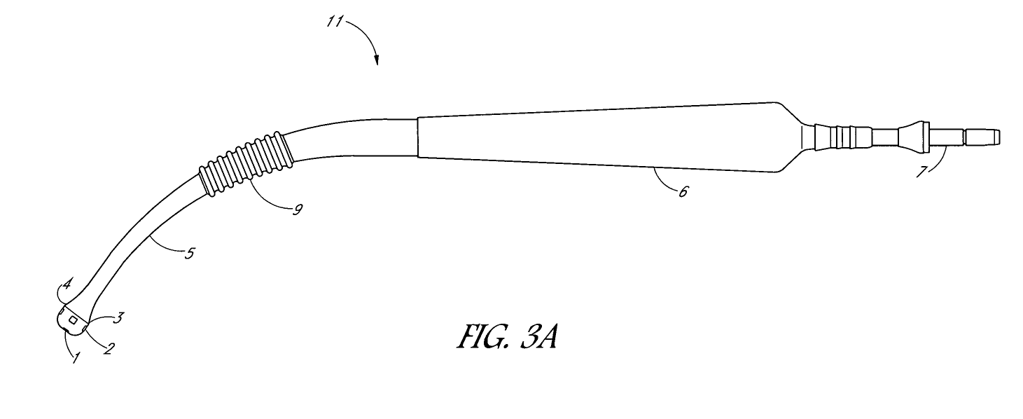

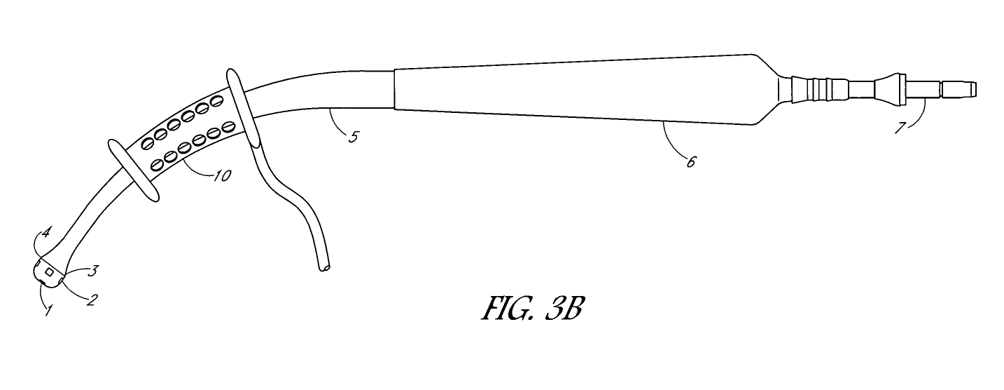

Referring to FIG. 3A, in another embodiment of the oral irrigation/fluid delivery device 11, an optional shock absorbing, flexible, bendable etc. section 9 could be made to the device's shaft 5. This modification would make the use of the device safer for children under the age of 6 years old, or for users who may have impairments that would make the use of sharp-tipped devices for oral feeding or irrigation not recommended. Further, this same option of including the device's bulbous tip 3 affixed to a shaft 5 that is then incorporated into the straw tube of a water bottle (like those used for athletes etc.) or the tip end of a syringe, making them safer for use and avoiding the possible damage that sharper tubes or straws may make to the inside of the mouth or surgical area following a medical procedure.

Referring to FIG. 3B, in another embodiment of the oral irrigation/fluid delivery device 11, is an optional fluid recovery/redirection section 10 could be made to the device's shaft 5. This modification would allow the user to purse their lips around the fluid recovery section 10 and blow the excess fluid out through the section to its attached tube which would direct the fluid to a basin sink etc. The advantage of this fluid recovery capability is that for those with physical impairments who may wish to use the device in a location that is not “over a sink” or basin, they could be using the fluid recovery section 10 and its attached tube to collect the excess fluid used during the rinsing procedure.

Referring to FIGS. 1 through FIGS. 3, in another embodiment of the oral irrigation/fluid delivery device 11, is an optional illumination system that would illuminate the device's shaft 5. This modification to the handle 6 would allow for light to travel the plastic shaft causing it to glow. This ability could be used to make the device more user friendly for children or to aid in the illumination of a user's mouth.

It should be readily appreciated by those skilled in the art that modifications and variations can be made to the embodiments of the invention described herein. It is intended that the invention include such modifications as come within the scope and spirit of the invention as set forth in the appended claims.

What is claimed is:

1. A oral irrigation/fluid delivery device, comprising:
an elongated wand tube having an irrigation tip comprised of a smooth bulbous end with four or more equally spaced orifices/holes around the perimeter of the bulbous end and one orifice/hole in the center of the bulbous end at a distal end thereof, said irrigation wand tube having a proximal end that is connectable to a liquid delivery source or pump;
an option to build the bulbous end of rubber for further safety measures;
an option to build the bulbous end of the device in a smooth, non-damaging design;
an expansion chamber in the handle of the oral irrigation device wand to reduce the pressure and pulsing action of the liquid that is being pumped in from an optional oral irrigation pump;
an optional shock absorbing or flexible shaft to minimize potential damage if said device was pushed into the mouth too hard or the user lacked the manual dexterity to safely use said device;
an optional no-drip collar that attaches to the shaft of the device or is used in conjunction with the incorporation of the device's bulbous tip;
an optional to incorporation of the device's bulbous tip only to a straw device for water bottles or attachment to a syringe or other irrigation or fluid delivery tips;
an optional set of connecting ends that allow the device to be connected to commercially available water pumps such as those provided by WaterPik™ and others;
an optional connecting end for the device to allow its connection to a syringe;
an optional connecting end for the device to allow its connection to a water supply from a faucet etc.;
an optional integration of the device with water bottles replacing the sharp tube-like straws providing a safer and more comfortable means of using said bottles;
an optional venting structure with an attached tube that could capture excess fluid etc. to allow the fluid to be “blown out” while the device is in the mouth;
an optional “decorative” or esthetic design feature including possible light sources to make the device attractive to children and or to provide a light source to “see” inside the mouth while the device is being used;

2. The irrigation device as in claim 1, further comprising a handle member at said proximal end of said irrigation wand tube, said handle member comprising an adapter for connecting said irrigation device to a fluid delivery source. The same bulbous tip design of the device can be incorporated onto other “tube like” devices such as straws, syringes etc.

3. The device's bulbous tip design with optional no-drip collar, optional flexible/shock absorbing shaft could be incorporated into syringe tips or water bottles. Any combination of said device could be incorporated in part of full.
